SEQUENCE LISTING (1 sequence)

SEQ ID NO: 1 - protein, 403 aa, human [sequence omitted]

(19) United States
(12) Patent Application Publication
(10) Pub. No.: US 2010/0303809 A1
(43) Pub. Date: Dec. 2, 2010

(54) METHODS FOR THE DETECTION AND QUANTITATION OF PTEN

(75) Inventor: Sarah S. Bacus, Hinsdale, IL (US)

Correspondence Address: K&L Gates LLP, P.O. Box 1135, CHICAGO, IL, 60690, US

(73) Assignee: TARGETED MOLECULAR DIAGNOSTICS, LLC, Westmont, IL (US)

(21) Appl. No.: 12/791,569

(22) Filed: Jun. 1, 2010

Related U.S. Application Data
(60) Provisional application No. 61/182,914, filed on Jun. 1, 2009.

(51) Int. Cl. A61K 39/395; A61K 31/5377; A61K 31/517; A61P 35/00; C12Q 1/34; G01N 33/573

(52) U.S. Cl. 424/133.1; 424/174.1; 424/143.1; 514/234.5; 514/266.4; 514/266.24; 435/18; 435/7.4

(57) ABSTRACT

The present disclosure provides methods for determining if PTEN is elevated or reduced in one or more tumor cells relative to one or more normal cells in the same biological sample by obtaining a biological sample comprising one or more tumor cells and one or more normal cells; assaying the biological sample for expression of PTEN; quantitating an amount of PTEN expression in the one or more tumor cells and an amount of PTEN expression in the one or more normal cells; comparing the amount of PTEN expression in the tumor cells to the amount of PTEN expression in the normal cells; and determining that PTEN is elevated in the tumor cells where the amount of expression of PTEN is greater in the tumor cells as compared to the normal cells or determining that PTEN is reduced in the tumor cells where the amount of expression of PTEN is less in the tumor cells than in the normal cells. Such methods may be used to predict whether a patient will be responsive to treatment with one or more receptor tyrosine kinase inhibitors and/or may be used to select subjects for inclusion/exclusion in a clinical trial.

### BACKGROUND

The epidermal growth factor receptor (EGFR) family comprises four closely related receptors including, for example, HER1/EGFR, HER2, HER3 and HER4 (“HER family members”) that are involved in cellular responses such as differentiation and proliferation. HER family members are typically involved in stimulating signaling pathways that promote multiple processes that are potentially cancer-promoting (e.g. proliferation, angiogenesis, cell motility and invasion, decreased apoptosis and induction of drug resistance). As such, over-expression of Her family members are frequently associated with many cancers including, for example, breast, lung, colorectal, ovarian, renal cell, bladder, head and neck cancers, glioblastomas, and astrocytomas.

PTEN (phosphatase and tensin homologue deleted on chromosome ten)/MMAC (mutated in multiple advanced cancers) phosphatase is a tumor suppressor that is mutated or deleted in a wide variety of human cancers. Notably, PTEN is a phosphatase that modifies other proteins and fats (e.g., lipids) by removing phosphate groups. Unlike most phosphatases that dephosphorylate tyrosine or serine/threonine kinases, the main substrates of PTEN are inositol phospholipids generated by the activation of the phosphoinositide 3 kinase (PI3K). The inositol phospholipids generated by PI3K lead to downstream activation of Akt, mTOR and ultimately cell survival and protein translation. Therefore, PTEN is an important negative regulator of PI3K/Akt signaling and plays a critical role in tumor suppression. Deletion or mutation or lack of PTEN expression has been shown to contribute to resistance to Herceptin® and erlotinib (Berns et al. (2007) Cancer Cell 12, 395-402; Kobayashi et al. (2005) N Engl J Med 352, 786-792).

Several Her family antagonists have been shown to offer clinical benefit including, for example, erlotinib, gefitinib and lapatinib. Anti-EGFR antibodies have also shown clinical utility, including cetuximab and panitumamab which are approved for the treatment of EGFR-expressing, metastatic colorectal carcinoma. A breakthrough in the field of EGFR-targeted therapy occurred in 2004 with the identification of somatic mutations in the EGFR gene, which were closely associated with a favorable clinical response to gefitinib and erlotinib treatment in NSCLC patients. These genetic alterations consisted of small in-frame deletions or point mutations in EGFR exons 18-24, which encode the kinase domain of the protein and are clustered in two mutational ‘hot spots’ in the EGFR gene. Given that the levels of PTEN in tumor cells may affect tumor response to a particular therapeutic, a quantitative evaluation of PTEN levels may represent an important prognostic/diagnostic marker.

### SUMMARY

The present disclosure provides methods for the detection and quantitation of PTEN in a biological sample (e.g., a formalin fixed paraffin embedded tissue sample) including, for example, methods for quantitating PTEN expression in one or more tumor cells relative to PTEN expression in one or more stromal cells derived from the same biological sample.

The present disclosure also provides methods for determining if PTEN is elevated or reduced in one or more tumor cells relative (e.g., compared to) to one or more normal cells in a biological sample by obtaining the biological sample comprising one or more tumor cells and one or more normal cells; assaying the biological sample for expression of PTEN; quantitating an amount of PTEN expression in the one or more tumor cells and an amount of PTEN expression in the one or more normal cells; comparing the amount of PTEN expression in the tumor cells to the amount of PTEN expression in the normal cells; and determining that PTEN is elevated in the tumor cells where the amount of expression of PTEN is greater in the tumor cells as compared to the normal cells or determining that PTEN is reduced in the tumor cells where the amount of expression of PTEN is less in the tumor cells than in the normal cells.

The present disclosure also provides methods for predicting responsiveness of a subject with a disease or disorder to a receptor tyrosine kinase inhibitor by obtaining the biological sample comprising one or more tumor cells and one or more normal cells from the subject; assaying the biological sample for expression of PTEN; quantitating an amount of PTEN expression in the one or more tumor cells and an amount of PTEN expression in the one or more normal cells; comparing the amount of PTEN expression in the tumor cells to the amount of PTEN expression in the normal cells; determining that PTEN is elevated in the tumor cells where the amount of expression of PTEN is greater in the tumor cells as compared to the normal cells or determining that PTEN is reduced in the tumor cells where the amount of expression of PTEN is less in the tumor cells than in the normal cells; and predicting that the subject is responsive to a receptor tyrosine kinase inhibitor where expression of PTEN in the tumor cells is less than expression of PTEN in the normal cells or determining that the subject is not responsive to a receptor tyrosine kinase inhibitor where expression of PTEN in the tumor cells is the same or greater than expression of PTEN in the normal cells.

The present disclosure also provides methods for treating a subject with a disease or disorder with a receptor tyrosine kinase inhibitor by obtaining the biological sample comprising one or more tumor cells and one or more normal cells from the subject; assaying the biological sample for expression of PTEN; quantitating an amount of PTEN expression in the one or more tumor cells and an amount of PTEN expression in the one or more normal cells; comparing the amount of PTEN expression in the tumor cells to the amount of PTEN expression in the normal cells; determining that PTEN is elevated in the tumor cells where the amount of expression of PTEN is greater in the tumor cells as compared to the normal cells or determining that PTEN is reduced in the tumor cells where the amount of expression of PTEN is less in the tumor cells than in the normal cells; and administering the receptor tyrosine kinase inhibitor to the subject.

In an embodiment of any of the above-described methods, the normal cells are stromal cells.

In an embodiment of any of the above-described methods, PTEN is mutated (e.g., a substitution or deletion).

In an embodiment of any of the above-described methods, the biological sample is a tumor biopsy. In an embodiment of any of the above-described methods, the biological sample is an aspirate.

In an embodiment of any of the above-described methods, the normal and tumor cells are assayed using a detectably labeled antibody or binding fragment thereof specific for PTEN. In an embodiment of any of the above-described methods, the antibody is a monoclonal antibody. In an embodiment of any of the above-described methods, the label is a chromagen or fluorophore.

In an embodiment of any of the above-described methods, the step of assaying is performed by immunohistochemistry (IHC) or western blot.

In an embodiment of any of the above-described methods, the step of quantitating PTEN expression in the normal cells and the step of quantitating PTEN expression in the tumor cells is preformed by image analysis.

In an embodiment of any of the above-described methods, the step of quantitating PTEN expression in the normal cells and the tumor cells is preformed in a defined cellular area. In an embodiment of any of the above-described methods, the defined cellular area is the nucleus. In an embodiment of any of the above-described methods, the defined cellular area is the cytoplasm. In an embodiment of any of the above-described methods, the defined cellular area is the membrane.

In an embodiment of any of the above-described methods, the amount of PTEN expression for the normal cells and the tumor cells is quantitated from an average optical density (OD) of PTEN per pixel of the defined cellular area.

In an embodiment of any of the above-described methods, the amount of PTEN expression for the normal cells and the tumor cells is quantitated from an average OD determined for the tumor cells and the normal cells on a per cell basis in the defined cellular area. In an embodiment of any of the above-described methods, the average OD on a per cell basis is obtained by dividing the average OD for the defined cellular area by a number of nuclei in the defined cellular area.

In an embodiment of any of the above-described methods, the average optical density is determined by using image analysis.

In an embodiment of any of the above-described methods, PTEN comprises a amino acid sequence as set forth in SEQ ID NO: 1. Alternatively, PTEN comprises a variant (e.g., a biologically active variant) of the amino acid sequence as set forth in SEQ ID NO: 1.

In an embodiment of any of the above-described methods, the receptor tyrosine kinase inhibitor is an antibody. In an embodiment of any of the above-described methods, the antibody is a monoclonal antibody. In an embodiment of any of the above-described methods, the monoclonal antibody is cetuximab (Erbitux), panitumumab, zalutumumab, nimotuzumab or matuzumab.

In an embodiment of any of the above-described methods, the receptor tyrosine kinase inhibitor is a small molecule inhibitor. In an embodiment of any of the above-described methods, the small molecule inhibitor is gefitinib, erlotinib or lapatinib.

In an embodiment of any of the above-described methods, the disease or disorder is cancer. In an embodiment of any of the above-described methods, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In an embodiment of any of the above-described methods, the subject is a cancer patient.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 shows a comparison of three PTEN antibodies by western blot analysis. Cell lysates were prepared from a panel of cell lines that represent different PTEN status (shown in D). (A.) PTEN mouse monoclonal antibody (Cascade #ABM-2052). (B.) PTEN mouse monoclonal antibody (NovoCastra #NCL-PTEN). (C.) PTEN rabbit monoclonal antibody (clone 138G6) (Cell Signaling #M9559). (D.) Table summarizing the PTEN allelic status of the panel of 6 cell lines used for the validation.

FIG. 2 shows IHC staining for PTEN of formalin fixed paraffin embedded cell pellets. The panel of 6 cell lines was stained for PTEN using the antibody at a 1:50 dilution.

FIG. 3 demonstrates the reproducibility of the PTEN IHC Assay. (A.) Three serial sections from a lung cancer specimen were stained for PTEN on 3 different days on an automated stainer. (B.) Image analysis was used to measure the sum of optical density of PTEN staining in both the tumor cells and stromal cells of each section.

FIG. 4 shows lung cancer tissue formalin-fixed, paraffin-embedded and stained for PTEN by IHC. The tissue specimen was analyzed by an Aperio ScanScope. Average OD was calculated by measuring total OD of a defined area of the tissue and dividing by the number of cell nuclei within the area to determine average OD per cell. Areas analyzed consisted of either pure tumor or pure normal stromal tissue as defined by an anatomical pathologist.

FIG. 5 shows lung cancer tissue formalin-fixed, paraffin-embedded and stained for PTEN by IHC. The tissue specimen was analyzed by an Aperio ScanScope. Average OD was calculated by measuring total OD of a defined area of the tissue and dividing by the number of cell nuclei within the area to determine average OD per cell. Areas analyzed consisted of either pure tumor or pure normal stromal tissue as defined by an anatomical pathologist.

FIG. 6 shows a PTEN FISH assay (Vysis) that was performed on a FFPE breast cancer sample. Red probe is specific for PTEN and the green probe is specific for the centromere for chromosome 10 where PTEN is encoded. The above pictures reveal that the normal surrounding tissue has full compliment of PTEN gene as indicated by the red fluorescent punctate dots (left panel), while the tumor containing tissue (right panel) shows the presence of the PTEN containing chromosome indicated by green fluorescence, but has low to no presence of the PTEN gene indicative of loss of red fluorescence punctate dots.

FIG. 7 shows that the FFPE breast tumor tissue that was tested for PTEN FISH and showed a demonstrable loss in PTEN gene presence in the tumor tissue (see FIG. 6), also shows PTEN loss by IHC compared to surrounding normal fibroblasts (indicated by white arrows above). This loss in PTEN expression correlates with loss in PTEN gene. With the ease and quanitifiability of IHC, the inventors have demonstrated that PTEN IHC can be performed in lieu of PTEN FISH as a biomarker for patient tumors.

### DETAILED DESCRIPTION

PTEN is one of the most commonly lost tumour suppressors in human cancer. During tumor development, mutations and deletions of PTEN occur that inactivate its enzymatic activity leading to increased cell proliferation and reduced cell death. Frequent genetic inactivation of PTEN occurs in glioblastoma, endometrial cancer, prostate cancer, and reduced expression is found in many other tumor types such as lung and breast cancer. Several recent clinical studies have shown that expression of PTEN is a significant predictor of resistance to treatment with receptor tyrosine kinase inhibitors including, for example, EGFR inhibitors. As such, assays have been developed to detect PTEN including, for example, the presence/absence of PTEN polynucleotide and/or expression of PTEN protein in a patient sample. These assays may detect PTEN by several methods including, for example, fluorescence in situ hybridization (FISH), PTEN promoter methylation and sequencing of PTEN. Such assays often compare PTEN expression in a biological sample (e.g., a tumor sample) obtained from a patient to that of a biological sample obtained from a control subject (e.g., a subject that does not have a tumor). However, baseline PTEN expression may vary from individual to individual and from tissue to tissue. Accordingly, comparing PTEN expression from a sample obtained from a patient to that of a control subject or sample may lead to an erroneous assessment of whether PTEN levels (e.g., protein levels) are in fact elevated/reduced in the patient. Thus, methods to detect PTEN expression relative to normal cells derived from the same biological sample are desired. Surprisingly, the inventors have discovered an immunohistochemistry (IHC) based-assay that can accurately and reliability determine the expression level of PTEN in a biological sample including, for example, a formalin fixed paraffin embedded tissue. This assay relies upon the determination of the relative amount of PTEN in control cells (e.g., stromal cells) versus tumor cells obtained from the same biological sample (e.g., same patient and/or same tissue). Remarkably, the methods of the present disclosure have near one-hundred percent concordance with FISH and sequencing based methods used to detect PTEN expression. Additionally, the methods of the present disclosure may be used to predict whether a patient will be responsive to treatment with one or more receptor tyrosine kinase inhibitors and may be used to select subjects for inclusion/exclusion in a clinical trial.

The present disclosure provides methods for determining if PTEN expression levels (e.g., protein and/or mRNA expression) are elevated or reduced in one or more tumor cells in a biological sample by obtaining the biological sample comprising one or more tumor cells and one or more stromal cells; assaying the biological sample for expression of PTEN by using a detectably labeled binding molecule specific for PTEN; quantitating an amount of PTEN expression in the one or more tumor cells and an amount of PTEN expression in the one or more stromal cells by determining an optical density (OD) reading (e.g., an OD value) for PTEN expression in the tumor cells and an OD reading for PTEN expression in the normal cells, wherein the OD reading for the tumor cells and stromal cells is quantitated from an average OD reading determined for the tumor cells and the normal cells on a per cell basis in a defined cellular area and wherein the average OD reading on a per cell basis is obtained by dividing the average OD reading for the defined cellular area by a number of nuclei in the defined cellular area; comparing the PTEN OD reading in the tumor cells to the PTEN OD reading in the stromal cells; and determining that PTEN is elevated in the tumor cells where the PTEN OD reading is greater in the tumor cells as compared to the stromal cells or determining that PTEN is reduced in the tumor cells where the PTEN OD reading is less in the tumor cells than in the stromal cells.

The present disclosure provides methods for determining if PTEN is elevated or reduced in one or more tumor cells relative to (e.g., compared to) one or more normal cells in a biological sample (e.g., a formalin fixed paraffin embedded tissue) by obtaining the biological sample comprising one or more tumor cells and one or more normal cells; assaying the biological sample by an immunohistochemistry (IHC) based technique for expression of PTEN using an antibody specific for PTEN; quantitating an amount of PTEN expression in the one or more tumor cells and an amount of PTEN expression in the one or more normal cells; comparing the amount of PTEN expression in the tumor cells to the amount of PTEN expression in the normal cells; and determining that PTEN is elevated in the tumor cells where the amount of expression of PTEN is greater in the tumor cells as compared to the normal cells or determining that expression of PTEN is reduced in the tumor cells where the amount of expression of PTEN is less in the tumor cells than in the normal cells.

The present disclosure also provides methods for selecting subjects for inclusion/exclusion in a clinical trial by obtaining a biological sample from each subject comprising one or more tumor cells and one or more stromal cells; assaying the biological sample for expression of PTEN by using a detectably labeled binding molecule specific for PTEN; quantitating an amount of PTEN expression in the one or more tumor cells and an amount of PTEN expression in the one or more stromal cells (e.g., by determining an average optical density (OD) reading for the tumor cells and for the stromal cells; comparing the amount of PTEN expression in the tumor cells to the amount of PTEN expression in the stromal cells; determining that PTEN levels are elevated in the tumor cells where the amount of PTEN expression is greater in the tumor cells as compared to the stromal cells or determining that PTEN is reduced in the tumor cells where the amount of PTEN expression is less in the tumor cells than in the stromal cells; and selecting subjects for inclusion/exclusion in the clinical trial which have an amount of PTEN expression that is greater in the tumor cells as compared to the stromal cells or selecting subjects for inclusion/exclusion in the clinical trial which have an amount of PTEN expression that is less in the tumor cells as compared to the stromal cells. In some embodiments, subjects are selected for inclusion in the clinical trial that have an amount of PTEN expression that is greater in the tumor cells as compared to the stromal cells. In some embodiments, subjects are selected for exclusion from the clinical trial that have an amount of PTEN expression that is greater in the tumor cells as compared to the stromal cells. In some embodiments, subjects are selected for inclusion in the clinical trial that have an amount of PTEN expression that is equal to or less than in the tumor cells as compared to the stromal cells. In some embodiments, subjects are selected for exclusion from the clinical trial that have an amount of PTEN expression that is equal to or less than in the tumor cells as compared to the stromal cells.

The present disclosure also provides methods for predicting responsiveness of a subject with a disease or disorder to a receptor tyrosine kinase inhibitor by obtaining the biological sample (e.g., a formalin fixed paraffin embedded tissue) comprising one or more tumor cells and one or more normal cells from the subject; assaying the biological sample by an immunohistochemistry (IHC) based technique for expression of PTEN using an antibody specific for PTEN; quantitating an amount of PTEN expression in the one or more tumor cells and an amount of PTEN expression in the one or more normal cells; comparing the amount of PTEN expression in the tumor cells to the amount of PTEN expression in the normal cells; determining that PTEN is elevated in the tumor cells where the amount of expression of PTEN is greater in the tumor cells as compared to the normal cells or determining that expression of PTEN is reduced in the tumor cells where the amount of expression of PTEN is less in the tumor cells than in the normal cells; and predicting that the subject is responsive to a receptor tyrosine kinase inhibitor where expression of PTEN in the tumor cells is less than expression of PTEN in the normal cells or determining that the subject is not responsive to a receptor tyrosine kinase inhibitor where expression of PTEN in the tumor cells is the same or greater than expression of PTEN in the normal cells.

The present disclosure also provides methods for treating a subject with a disease or disorder with a receptor tyrosine kinase inhibitor by obtaining the biological sample (e.g., a formalin fixed paraffin embedded tissue) comprising one or more tumor cells and one or more normal cells from the subject; assaying the biological sample by an immunohistochemistry (IHC) based technique for expression of PTEN using an antibody specific for PTEN; quantitating an amount of PTEN expression in the one or more tumor cells and an amount of PTEN expression in the one or more normal cells; comparing the amount of PTEN expression in the tumor cells to the amount of PTEN expression in the normal cells; determining that PTEN is elevated in the tumor cells where the amount of expression of PTEN is greater in the tumor cells as compared to the normal cells or determining that expression of PTEN is reduced in the tumor cells where the amount of expression of PTEN is less in the tumor cells than in the normal cells; and administering the receptor tyrosine kinase inhibitor to the subject.

PTEN may comprise the amino acid sequence as set forth in SEQ ID NO: 1. Alternatively, PTEN may be a variant including, for example, a biologically active variant, of the amino acid sequence as set forth in SEQ ID NO: 1.

Variants of PTEN may include biologically active variants which comprise an amino acid sequence that is at least 80%, more preferably 90%, still more preferably 95-99% similar to the native protein.

Guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

Protein variants include glycosylated forms, aggregative conjugates with other molecules, and covalent conjugates with unrelated chemical moieties. Also, protein variants also include allelic variants, species variants, and muteins. Truncations or deletions of regions which do not affect the differential expression of the gene are also variants. Covalent variants can be prepared by linking functionalities to groups which are found in the amino acid chain or at the N- or C-terminal residue, as is known in the art.

It will be recognized in the art that some amino acid sequence of PTEN can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Thus, the polypeptides of the present invention may include one or more amino acid substitutions, deletions or additions, either from natural mutations or human manipulation.

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255: 306-312 (1992)).

Variants of PTEN may include a protein possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to PTEN (SEQ ID NO: 1). Preferably, this variant may possess at least one biological property in common with the native protein.

Sequence identity or percent identity is intended to mean the percentage of the same residues shared between two sequences, when the two sequences are aligned using the Clustal method [Higgins et al, Cabios 8:189-191 (1992)] of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 [Dayhoff et al. in Atlas of Protein Sequence and Structure, Dayhoff, Ed., NDRF, Washington, Vol. 5, suppl. 3, p. 345, (1978)].

In one embodiment, the disease or disorder may be cancer. In one embodiment the cancer may be selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, glioma; parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In another embodiment the cancer may be non-small cell lung cancer, pancreatic cancer, breast cancer, ovarian cancer, colorectal cancer, or head and neck cancer. In yet another embodiment the cancer may be a carcinoma, a tumor, a neoplasm, a lymphoma, a melanoma, a glioma, a sarcoma, or a blastoma.

In one embodiment the carcinoma may be selected from the group consisting of: carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiated carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In another embodiment the tumor may be selected from the group consisting of: astrocytic tumors, malignant mesothelial tumors, ovarian germ cell tumors, supratentorial primitive neuroectodermal tumors, Wilms tumors, pituitary tumors, extragonadal germ cell tumors, gastrinoma, germ cell tumors, gestational trophoblastic tumors, brain tumors, pineal and supratentorial primitive neuroectodermal tumors, pituitary tumors, somatostatin-secreting tumors, endodermal sinus tumors, carcinoids, central cerebral astrocytoma, glucagonoma, hepatic adenoma, insulinoma, medulloepithelioma, plasmacytoma, vipoma, and pheochromocytoma.

In yet another embodiment the neoplasm may be selected from the group consisting of: intraepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma. In a further embodiment the lymphoma may be selected from the group consisting of: nervous system lymphoma, AIDS-related lymphoma, cutaneous T-cell lymphoma, non-Hodgkin's lymphoma, lymphoma, and Waldenstrom's macroglobulinemia. In another embodiment the melanoma may be selected from the group consisting of: acral lentiginous melanoma, superficial spreading melanoma, uveal melanoma, lentigo maligna melanomas, melanoma, intraocular melanoma, adenocarcinoma nodular melanoma, and hemangioma. In yet another embodiment the sarcoma may be selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma. In one embodiment the glioma may be selected from the group consisting of: glioma, brain stem glioma, and hypothalamic and visual pathway glioma. In another embodiment the blastoma may be selected from the group consisting of: pulmonary blastoma, pleuropulmonary blastoma, retinoblastoma, neuroblastoma, medulloblastoma, glioblastoma, and hemangiblastomas.

### Detection and Quantitation of PTEN

A number of methodologies may be employed to detect and/or quantitate the amount (i.e., level) of PTEN expression in a biological sample. Such expression of PTEN may be detected at the protein level and/or nucleic acid level. Those skilled in the art will appreciate that the methods indicated below represent some of the preferred ways in which the level of PTEN expression may be detected and/or quantitated and in no manner limit the scope of methodologies that may be employed. Those skilled in the art will also be able to determine operative and optimal assay conditions for each determination by employing routine experimentation. Such methods may include but are not limited to Western blots, ELISA, immunoprecipitation, immunofluorescence, flow cytometry, northern blots, PCR and immunocytochemistry (IHC). In a preferred embodiment, expression of PTEN may be detected and quantitated by IHC. Such methods of the present disclosure may comprise the detection and quantitation of the amount of the PTEN in a biological sample. PTEN may comprise the amino acid sequence as set forth in SEQ ID NO: 1. Alternatively, PTEN may be a variant of the amino acid sequence as set forth in SEQ ID NO: 1 and defined herein.

Biological samples that may be used in the methods of the present disclosure may include tissues, cells, biological fluids and isolates thereof, isolated from a subject, as well as tissues, cells and fluids present within a subject (e.g., a patient). Preferably, biological samples comprise cells, most preferably tumor cells, that are isolated from body samples, such as, but not limited to, smears, sputum, biopsies, secretions, cerebrospinal fluid, bile, blood, lymph fluid, urine and faeces, or tissue which has been removed from organs, such as breast, lung, intestine, skin, cervix, prostate, and stomach. Biological samples may also include sections of tissues such as frozen sections taken for histological purposes.

In some embodiments, PTEN expression may be quantitated by image analysis including, for example, computerized image analysis.

### Immunohistochemistry (IHC) Assays

The expression level of PTEN in a biological sample may be determined by immunohistochemically staining cells in the sample using a detectably-labeled agent (e.g., an antibody) specific for PTEN. In a preferred embodiment, the agent is a monoclonal antibody and the detectable label is a chromagen or a fluorophore.

PTEN can be detected using a specific agent, most preferably an antibody, that is itself detectably labeled, or using an unlabeled antibody specific for PTEN and a second antibody that is detectably labeled and recognizes the unlabeled antibody specific for PTEN. Alternatively, any molecule that can be detectably labeled and that specifically binds to PTEN can be used in the practice of the methods of the disclosure. In a preferred embodiment of the methods of the present disclosure, a two-component immunohistochemical staining system may be used to differentially stain PTEN and the tissue or cell sample so that the stained PTEN can be more readily distinguished from the counterstained tissue or cell sample.

In an exemplary method, PTEN in the biological sample may be identified by adding a detectably-labeled primary antibody specific for PTEN, or alternatively an unlabeled primary antibody and a detectably-labeled secondary antibody specific for the primary antibody. The antibodies are incubated with the sample for a time to form complexes if PTEN is present.

The complexes may then visualized by treating the sections with a stain such as diaminobenizidine (DAB) stain under appropriate conditions. In a second step, the tissue may be counterstained with another optical enhancement factor, for example ethyl green. Although a staining technique using peroxidase and ethyl green is exemplary, other stains and optical enhancement factors are also suitable such as alkaline phosphatase based with specific chromagens such as Fast Red, Fast Green, etc. For example, PTEN can be stained using diaminobenizidine (DAB) and the tissue or cell sample can be counterstained using ethyl green or methylene blue. Spectral studies have shown that the ethyl green stain offers good spectral separation from the DAB precipitate of the immunoperoxidase technique such that different features of the image can be readily separated by filtering it at two different wavelengths. This allows the image to be digitized into two separate images, one in which all the cell nuclei are optically enhanced (ethyl green or Fast Green) and one in which only those tissue areas with receptor staining (DAB) are optically enhanced. In a preferred embodiment, the images can be separated by a 600 nanometer (red) filter to produce an image of all of the counter stained area, and a 500 nanometer (green) filter to produce an image of only those tissue areas with the DAB precipitate staining.

In some embodiments, to further differentiate stained tissue areas, an interactive threshold setting technique can be used where an operator visualizing the images can set a boundary on the areas under consideration. When the boundaries are set, the images are formed by eliminating all parts of the image that are below the thresholds in optical density. In some embodiments, threshold may be set for the first image, and a second threshold may be set for the second image.

In some embodiments, where PTEN is quantitated by IHC, for example, using image analysis, the optical density (OD) threshold may be set at 0.5, 0.55, 0.60, 0.65, 0.70, 0.75, 0.80. 0.85, 0.90, 1.0 or greater.

In some embodiments, an OD threshold may be set such that those cellular areas (e.g., cell membrane, cytoplasm, or nucleus) that have staining below the threshold are eliminated from the final image on a per cell basis or on a per pixel basis.

The image processing method then consists of first forming the mask image of the tissues under consideration with the red filter. This mask image may be stored and another image for expressed protein quantification may then acquired by using the green filtered version of the same image. The effect of the filters in combination is to optically enhance (make darker) those areas of the tissue mask where tissue components are stained with DAB and to make lighter those tissue components with only green counterstain. An image analysis can then be performed using only those areas of the image that are stained and which are within the mask.

Red and green filters are suitable for practice of the disclosure as well as DAB and green counterstain. This implementation shows a convenient and advantageous method for discriminating between two areas having counterstaining. It is recognized that there are various other staining or optical enhancement methods and filtering methods which can be used to optically enhance one particular area or feature over another cell feature such as Fast green, eosin, and the like.

Following immunohistochemical staining, the optical image of the tissue or cell sample generated by the computer-aided image analysis system may then magnified under a light microscope and separated into a pair of images. Such equipment can include a light or fluorescence microscope, and image-transmitting camera and a view screen, most preferably also comprising a computer that can be used to direct the operation of the device and store and manipulate the information collected, most preferably in the form of optical density of certain regions of a stained tissue preparation. Image analysis devices useful in the practice of this disclosure include but are not limited to the CAS 200 (Becton Dickenson, Mountain View, Calif.), Chromavision or Tripath systems. The separated images may be enhanced using a pair of optical filters, one having a maximum absorption corresponding to the stain and the other having a maximum absorption corresponding to the counterstain. In other embodiments of the method of the present disclosure, a plurality of image analysis filters may be used to detect, differentiate, and quantitate the level of staining of different cellular proteins in various components (e.g., membrane, cytoplasm, and nucleus). In preferred embodiments, specific staining for PTEN may be detected, measured and quantitated using image analysis equipment, defined herein as comprising a light or fluorescence microscope, and image-transmitting camera and a view screen, most preferably also comprising a computer that can be used to direct the operation of the device and also store and manipulate the information collected, most preferably in the form of optical density of certain regions of a stained tissue preparation. Image analysis devices useful in the practice of this disclosure include but are not limited to the CAS 200 system (Becton Dickenson, Mountain View, Calif.). From a digitized image, a nuclear or cytoplasmic image mask may be formed by forming the image at one wavelength of light such as red wavelength or green optical filter. The tissue mask may be stored and a second filter may be used to form another filtered image of the areas with the optical enhancement factor. Differentiation of cellular characteristics can be made by comparing the first image with the second image to obtain a quantification of material stained with the optical enhancement factor and thus, an assay of the amount of the particular target under study.

After immunohistochemical staining, a quantified measure of the percentage of cells expressing PTEN can be taken by digitizing microscope images of stained samples, and converting light intensity values in each picture element (pixel) of the digitized image to optical density values, which correspond to the percentage of stained cell nuclei. More specifically, computerized image analysis can be used to determine from a digital grey scale image, a quantity of cells having a particular stain. The grey scale images are representative of the amount of an optical enhancement factor, such as a chromagen, which binds to a specific target under study and thereby allows optical amplification and visualization of the target.

The present disclosure also includes methods for fixing cells and tissue samples for analysis. Generally, neutral buffered formalin may be used. Any concentration of neutral buffered formalin that can fix tissue or cell samples without disrupting the epitope can be used. Preferably, the method includes suitable amounts of phosphatase inhibitors to inhibit the action of phosphatases and preserve phosphorylation. Any suitable concentration of phosphatase inhibitor can be used so long as the biopsy sample is stable and phosphatases are inhibited, for example 1 mM NaF and/or Na₃VO₄ can be used. In one method a tissue sample or tumor biopsy (e.g., biological sample) may be removed from a patient and immediately immersed in a fixative solution which can and preferably does contain one or more phosphatase inhibitors, such as NaF and/or Na₃VO₄. Preferably, when sodium orthovanadate is used it is used in an activated or depolymerized form to optimize its activity. Depolymerization can be accomplished by raising the pH of its solution to about 10 and boiling for about 10 minutes. The phosphatase inhibitors can be dissolved in the fixative just prior to use in order to preserve their activity. Fixed samples can then be stored for several days or processed immediately. To process the samples into paraffin after fixing, the fixative can be thoroughly rinsed away from the cells by flushing the tissue with water. The sample can be processed to paraffin according to normal histology protocols which can include the use of reagent grade ethanol. Samples can be stored in 70% ethanol until processed into paraffin blocks. Once samples are processed into paraffin blocks they can be analyzed histochemically for virtually any antigen that is stable to the fixing process.

In practicing the method of the present disclosure, staining procedures can be carried out by a technician in the laboratory. Alternatively, the staining procedures can be carried out using automated systems. In either case, staining procedures for use according to the methods of this disclosure are performed according to standard techniques and protocols well-established in the art.

The amount of PTEN can be quantitated by image analysis in the biological sample. For example, the amount of the expression of PTEN may be quantitated from an average optical density (OD) of expression of PTEN in a defined cellular area. Alternatively, the amount of the expression of PTEN may be quantitated from an average OD determined on a per cell basis in the defined cellular area. The average OD on a per cell basis may be obtained by dividing the average OD for the defined cellular area by a number of nuclei in the defined cellular area.

Three parameters may be established in order to validate an immunohistochemistry assay including, for example, sensitivity, specificity and/or reproducibility.

For sensitivity analysis, a known tissue type or cell line that expresses the target should be used to optimize the antibody dilution. For tissue, it may be useful if a particular cell type within the tissue expresses the target, while another particular cell does not (e.g. adrenal gland would be useful if a target was expressed in cortical cells but not medullar cells, or vice versa). For the assay to be acceptable, target localization should correspond to what may be stated in the package insert (if given), the accepted literature or other appropriate reference site.

For specificity analysis, the antibody should provide robust staining of the target in the appropriate tissue or cell type with minimal to no background staining of cell types or structures that do not express the target. If the antibody is suitable for western blotting, a cell line that expresses the target can be analyzed. The presence of a single band of the expected molecular weight of the target would indicate that the antibody is specific for the target. Even further proof of specificity, if possible, would be to treat the cells with an agent that modulates the expression of the target or identify a cell line that does not express the target at all. However, this is not always possible, especially if the target is highly or ubiquitously expressed (e.g. 4E-BP1).

For reproducibility analysis, inter-assay reproducibility may be evaluated by staining several positive tissues in 3 consecutive runs (usually performed on different days). Immunohistochemistry assays should be worked up on an automated staining system when possible (e.g. Dako Autostainer) to reduce variability.

### Protein Based Assays

The expression level of PTEN in a biological sample may be determined by immunohistochemically staining cells in the sample using a detectably-labeled agent (e.g., an antibody) specific for PTEN. PTEN expression may be quantified at the protein level using methods known in the art, for example using quantitative enzyme linked immunosorbent assays (“ELISA”). Methods for designing and using quantitative ELISA assays are well known in the art. These methods require use of monoclonal or polyclonal antibodies that are specific for the PTEN protein.

Suitable monoclonal antibodies may be prepared by standard hybridoma methods, using differential binding assays to ensure that the antibodies are specific for PTEN and do not show cross-reactivity between related proteins. Alternatively, suitable monoclonal antibodies may be prepared using antibody engineering methods such as phage display. Methods for obtaining highly specific antibodies from antibody phage display libraries are known in the art, and several phage antibody libraries are commercially available from, for example, MorphoSys (Martinsried, Germany), Cambridge Antibody Technology (Cambridge UK) and Dyax (Cambridge Mass.). Suitable phage display methods are described, for example, in U.S. Pat. Nos. 6,300,064 and 5,969,108; “Antibody Engineering,” McCafferty et al. (Eds.) (IRL Press 1996)). Once the antibody heavy and light chain genes are recovered from the phage antibodies, antibodies in any suitable format may be prepared, e.g. whole antibodies, Fab, scFv, etc.

Other antibody preparations may also be used, for example Camelid antibodies, which contain only heavy immunoglobulin chains (e.g., Muyldermans et al. J. Biotechnol. June; 74(4):277-302 (2001)). Other antibody formats are described, for example in “Antibody Engineering,” McCafferty et al. (Eds.) (IRL Press 1996).

Polyclonal antibodies specific for PTEN may also be prepared using traditional animal-based methods. Peptides derived from PTEN can be conjugated at their N- or C-termini to carrier proteins such as bovine serum albumin (BSA) or keyhole limpet hemocyanin (KLH) and used to immunize animals, such as rabbits, using well-known immunization regimes. Specific polyclonal antibodies can be obtained from the serum of the animal by, for example, affinity chromatography over a matrix containing the peptide used for immunization bound to a solid support.

In yet another embodiment of the present disclosure, expression of PTEN may be measured and quantitated by Western Blot analysis. Briefly, proteins samples may be electrophoresed on an acrylamide gel and transferred to a membrane such as nitrocellulose or PVDF. The blot may be detected with an antibody specific for PTEN. These primary antibodies may then be detected, for example, with labeled secondary antibodies. The fluorescence intensity of the dye may be measured for both a test and control sample and the ratio of the intensity indicates the ratio of the two proteins.

### Methods for Predicting Responsiveness to a Receptor Tyrosine Kinase Inhibitor

The present disclosure includes methods for predicting responsiveness of a subject with a disease or disorder to a receptor tyrosine kinase inhibitor by obtaining the biological sample (e.g., a formalin fixed paraffin embedded tissue) comprising one or more tumor cells and one or more normal cells from the subject; assaying the biological sample by an immunohistochemistry (IHC) based technique for expression of PTEN using an antibody specific for PTEN; quantitating an amount of PTEN expression in the one or more tumor cells and an amount of PTEN expression in the one or more normal cells; comparing the amount of PTEN expression in the tumor cells to the amount of PTEN expression in the normal cells; determining that PTEN is elevated in the tumor cells where the amount of expression of PTEN is greater in the tumor cells as compared to the normal cells or determining that expression of PTEN is reduced in the tumor cells where the amount of expression of PTEN is less in the tumor cells than in the normal cells; and predicting that the subject is responsive to a receptor tyrosine kinase inhibitor where expression of PTEN in the tumor cells is less than expression of PTEN in the normal cells or determining that the subject is not responsive to a receptor tyrosine kinase inhibitor where expression of PTEN in the tumor cells is the same or greater than expression of PTEN in the normal cells. The subject may be predicted to be responsive to the receptor tyrosine kinase inhibitor where the amount of PTEN detected and quantitated in the biological sample is less than the amount of PTEN detected in normal cells obtained from the same subject from which the biological sample was obtained or a different subject from which the biological sample was obtained.

In some embodiments, the subject may be predicted to be responsive to a receptor tyrosine kinase inhibitor where the amount of PTEN expressed in tumor cells from the biological sample is less than the amount of PTEN expressed in the normal cells from the biological sample. In some embodiments, the subject may be predicted to be responsive to a receptor tyrosine kinase inhibitor where the amount of PTEN expressed in tumor cells in the biological sample is 5%, 10%, 15%, 20%, 25%, 30%, 35%, 40%, 45%, 50%, 55%, 60%, 65%, 70%, 75%, 80%, 85%, 90%, 95% or 100% of the amount of PTEN detected in the normal cells in the biological sample. A subject may also be predicted to be responsive to the receptor tyrosine kinase inhibitor where the amount of PTEN expressed in tumor cells is above or below a set threshold. For example, a threshold may be set at the maximum amount of PTEN expressed in a tumor cells from a biological sample obtained from a subject where the subject is responsive to treatment with a receptor tyrosine kinase inhibitor. Such a threshold may be an average obtained from two or more subjects. Alternatively, the subject may be predicted to be responsive to a receptor tyrosine kinase inhibitor where the amount of PTEN expressed in tumor cells from the biological sample is more than the amount of PTEN expressed in the normal cells from the biological sample. In some embodiments, the subject may be predicted to be responsive to a receptor tyrosine kinase inhibitor where the amount of PTEN expressed in normal cells in the biological sample is 5%, 10%, 15%, 20%, 25%, 30%, 35%, 40%, 45%, 50%, 55%, 60%, 65%, 70%, 75%, 80%, 85%, 90%, 95%, 100% or more than the amount of PTEN detected in the tumor cells in the biological sample. A subject may also be predicted to be responsive to the receptor tyrosine kinase inhibitor where the amount of PTEN expressed in normal cells is above or below a set threshold.

In some embodiments, the receptor tyrosine kinase inhibitor may be an antibody including, for example, a monoclonal antibody. Monoclonal antibodies may include, but are not limited to cetuximab (Erbitux®), panitumumab, zalutumumab, nimotuzumab or matuzumab. In other embodiments, the receptor tyrosine kinase inhibitor is a small molecule inhibitor. Small molecule inhibitors may include, but are not limited to, gefitinib, erlotinib or lapatinib.

A determination of whether a subject will be predicted to be responsive to a receptor tyrosine kinase inhibitor may be used to direct a therapeutic regimen for a particular disease or disorder including, for example, cancer. Such methods may comprise obtaining the biological sample (e.g., a formalin fixed paraffin embedded tissue) comprising one or more tumor cells and one or more normal cells from the subject; assaying the biological sample by an immunohistochemistry (IHC) based technique for expression of PTEN using an antibody specific for PTEN; quantitating an amount of PTEN expression in the one or more tumor cells and an amount of PTEN expression in the one or more normal cells; comparing the amount of PTEN expression in the tumor cells to the amount of PTEN expression in the normal cells; determining that PTEN is elevated in the tumor cells where the amount of expression of PTEN is greater in the tumor cells as compared to the normal cells or determining that expression of PTEN is reduced in the tumor cells where the amount of expression of PTEN is less in the tumor cells than in the normal cells; and predicting that the subject is responsive to a receptor tyrosine kinase inhibitor where PTEN expression in the tumor cells is less than PTEN expression in the normal cells or determining that the subject is not responsive to a receptor tyrosine kinase inhibitor where PTEN expression in the tumor cells is the same or greater than PTEN expression in the normal cells.

This disclosure is further illustrated by the following examples which are provided to facilitate the practice of the disclosed methods. These examples are not intended to limit the scope of the disclosure in any way.

### EXAMPLES

### Example 1

### Detection of PTEN by Western Analysis

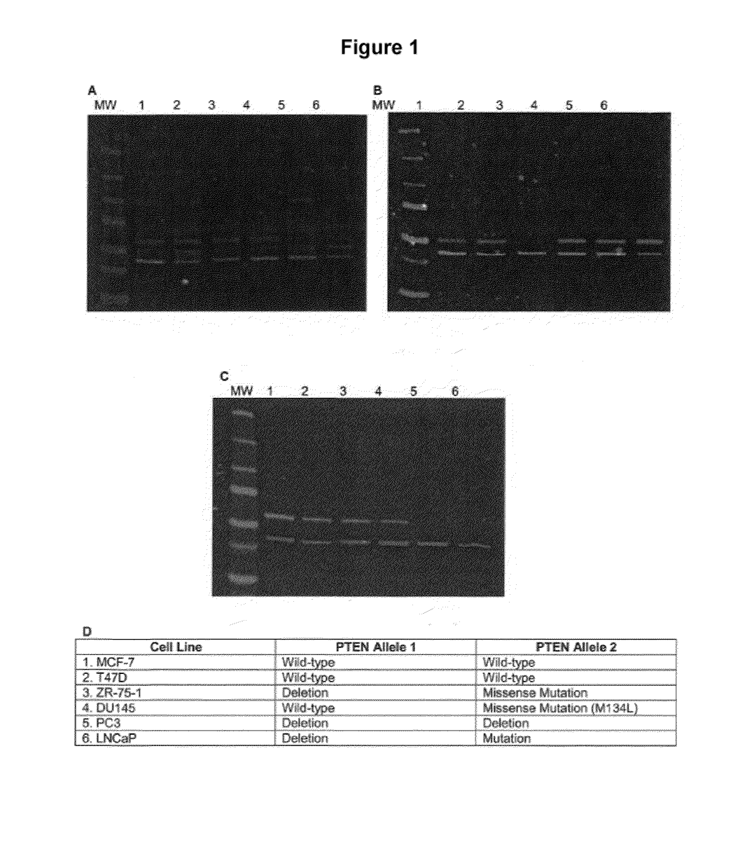

PTEN expression may be determined in a biological sample by any method know in the art including, for example, Western blot and IHC (see, e.g., FIG. 1).

In an exemplary method, T47D, DU145 and ZR-75-1 cells were grown in RPMI 1640 with 10% FBS and pen/strep/L-glut. PC3, LNCaP and MCF7 cells were grown in DMEM with 10% FBS and pen/strep/L-glut. All cells were maintained in a humidified atmosphere of 5% CO2 at 37° C. Cell lysates were prepared in modified RIPA buffer and 25 μg of each lysate was electrophoresed on either an 8% or 10% SDS polyacrylamide gel, transferred to PVDF membranes (Millipore, Billerica, Mass.) and blotted. The blots were then probed with a 1:250 dilution of the Cascade PTEN antibody, 1:500 dilution of the NovoCastra PTEN antibody and 1:500 dilution of the Cell Signaling PTEN antibody. All primary antibody incubations were done overnight in 5% BSA and 0.1% TBST at 4° C. Next, secondary antibodies LI-COR IRDye 800 (anti-mouse) and LI-COR IRDye680 (anti-rabbit) were incubated in a solution of 0.1% Tween 20 in 0.02% SDS for 1 hour at room temperature. Proteins were then visualized on a LICOR Odyssey.

### Example 2

### Preparation of Formalin Fixed Paraffin Embedded Cell Pellet

Agar was prepared in a 3% dilution, and 500 u1 aliquoted into 1.7 milliliter tubes. Cell suspension was removed from 4° C., spun down and washed 3 times with PBS. The pellet was resuspended in at least 500 μl of PBS to create the desired concentration. The cells were diluted 1:1 in a PBS/agar ratio. Tubes with agar were placed in the 10° C. heating block for 5 minutes to melt the gel. Tubes that contains agar were transferred to the 42° C. heating block. 500 μl of the cell suspension was transferred to a 1.7 mL tube and placed into the 42° C. heating block for 5 minutes.

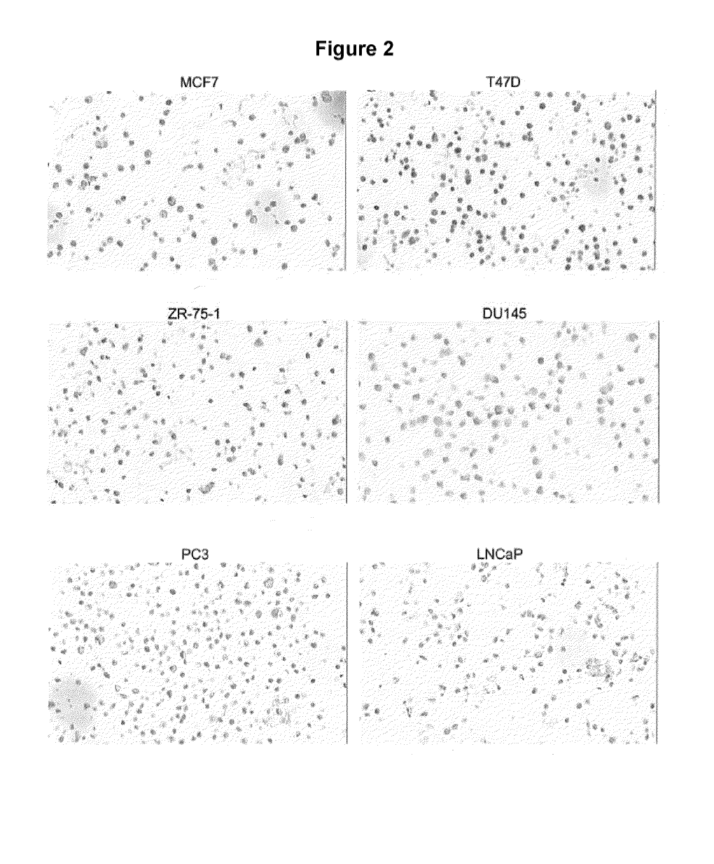

1000 μl tips were pre-warmed on the hot plate. The bottom of a petri dish was lined with parafilm and placed on top of the ice in the ice bucket. A drinking straw was cut into 2 cm lengths which were stood upright on top of the parafilm. The 500 μl of cell suspension was added to the 500 μl of agar. The solution was vortexed gently until it was visibly mixed. The 500 μl cell/agar mixture was pipetted into each straw piece without creating bubbles or knocking over the straw. Pellets were allowed to solidify for 10 minutes on ice. A pair of round nose tweezers or a glass thermometer was used to push the pellet out of the straw. The pellets were cut into 4 mm lengths and placed in a tissue cassette, one pellet per cassette. The cassettes were placed into a beaker on top of a stir plate with 10% NBF for at least 1 hour but not more that 24 hours. Pellets were washed in distilled water and placed into a beaker containing 70% ethanol and stirred with a stir bar on a stir plate for 1 to 24 hours. The pellets were then embedded into paraffin and sectioned. Paraffin embedded pellets were then stained for PTEN expression (see, FIG. 2).

### Example 3

### Detection of PTEN by IHC Staining

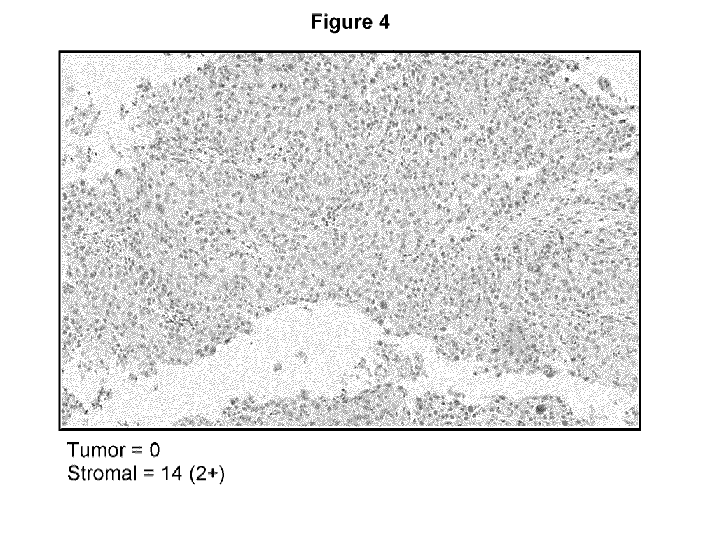

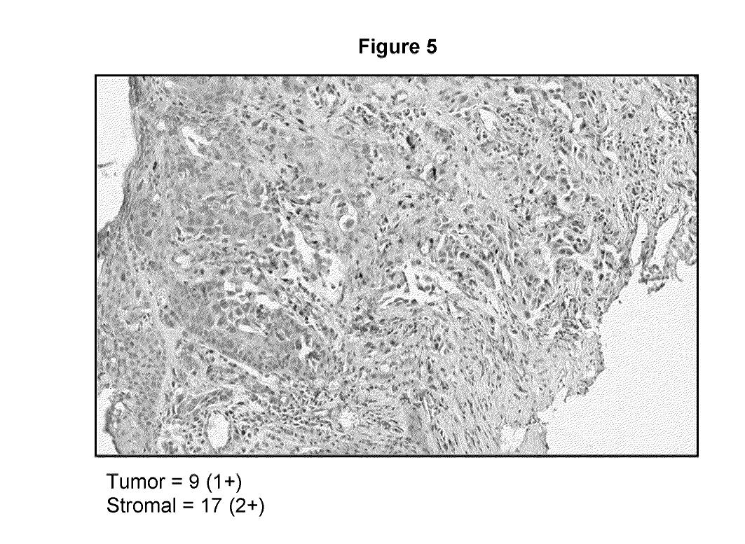

Lung tissue was used as positive control tissues to test the dilution and sensitivity of the antibody. An initial dilution range of 1:25, 1:50, 1:100 and 1:200 was chosen. A method negative control without the primary antibody was also run. Five tissue sections were sectioned, mounted on charged slides, deparaffinized and processed for antigen retrieval with citrate buffer, pH 6.0 (Dako, Carpinteria, Calif.) in a Decloaking Chamber (Biocare Medical, Concord, Calif.). Retrieval conditions were SP-1 for 30 seconds and SP-2 for 10 seconds at 125-128° C. Slides were loaded onto a Dako Autostainer, rinsed and incubated with 3% H202. Slides were then rinsed and then incubated with the primary PTEN antibody for 30 minutes at 24° C. Next, slides were rinsed and then incubated with the Envision+Dual Link Polymer (Dako) secondary detection system for 30 minutes at 24° C. Slides were then rinsed and then incubated with DAB+ (Dako) for 5 minutes at 24° C. Slides were rinsed a final time, removed from the Autostainer and counterstained with methyl green (Dako) (see, FIGS. 4 and 5). A board-certified pathologist analyzed and signed off on the optimal 1:25 dilution.

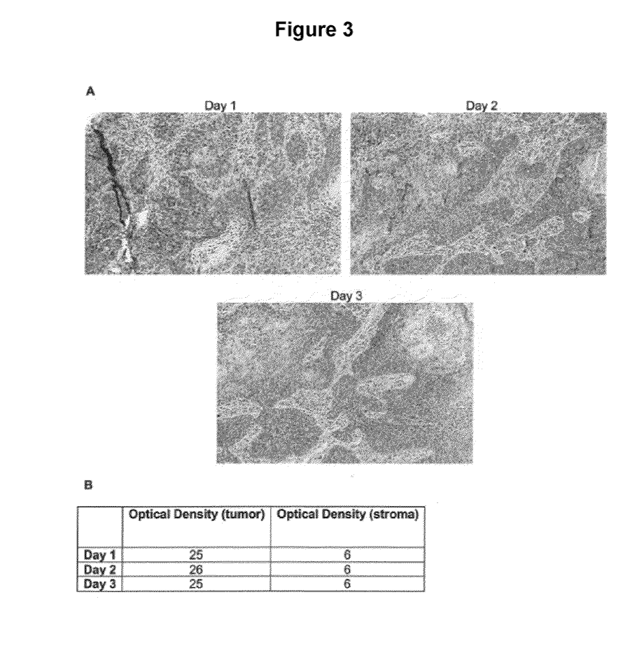

PTEN IHC staining was analyzed utilizing the Aperio Scanscope XT and Spectrum Image Analysis Software Version 9.0.746.1516. Three to 5 (on average) fields of stromal cells and 3- to 5 (on average) fields of tumor cells were analyzed for each specimen section. The sum of the optical density (OD) was measured for approximately equal numbers of tumor cells and stromal cells separately. A ratio of tumor cell PTEN OD to stromal cell PTEN OD provided the relative amount of tumor PTEN expression for each specimen. Results for PTEN were reported as OD units and as a manual pathologist score (0, 1+, 2+, 3+). The results of the IHC staining were reproducible as demonstrated by PTEN staining on 3 different days (see, FIG. 3).

The specificity of three PTEN antibodies using a panel of cell lines representing normal and mutant. PTEN alleles is shown in FIG. 1. The Cascade (FIG. 1, Panel A) and CST (FIG. 1) antibodies detected PTEN in wild-type cell lines (MCF7, T47D) but not in a cell line with a homozygous deletion (PC3). The CST antibody detected less background. The NovoCastra antibody (FIG. 1, Panel B) detected a band in PC3 cells and was not considered for further development. Cell lines with mutant alleles may or may not be detected (e.g., ZR-75-1 and LNCaP), most likely depending on the degree to which protein structure is affected by a particular mutation, and possibly other factors such as epigenetic silencing.

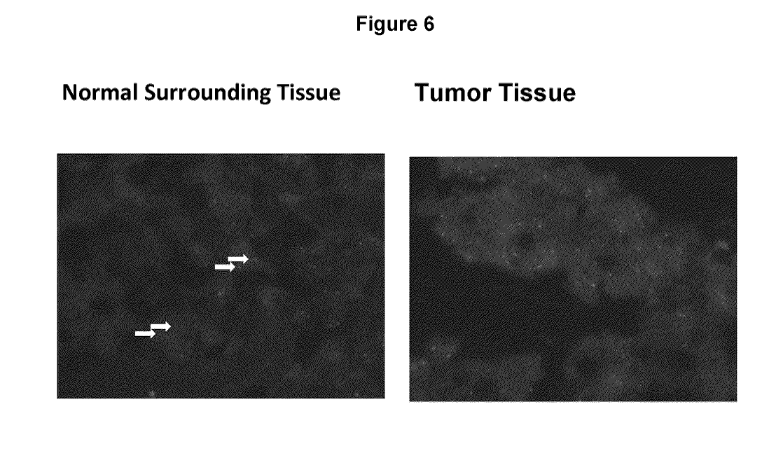

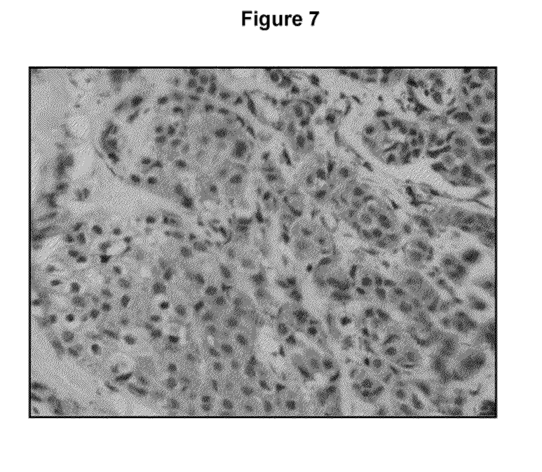

Moreover, PTEN IHC demonstrates that PTEN loss by FISH can be corroborated by PTEN loss measured by IHC. Briefly, PTEN FISH analysis was performed for two fields of the same patient sample and revealed loss of the PTEN gene in the tumor tissue (see FIG. 6). Notably, PTEN IHC on the same breast cancer sample also showed PTEN loss. Immunohistochemistry staining for PTEN demonstrated low expression in the tumor tissue with high expression in the surrounding normal fibroblasts (indicated by arrows) (see, FIG. 7).

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method for determining if PTEN expression is elevated or reduced in one or more tumor cells relative to one or more normal cells in a biological sample, said method comprising:
(a) obtaining the biological sample comprising one or more tumor cells and one or more normal cells;
(b) assaying the biological sample for expression of PTEN;
(c) quantitating an amount of PTEN expression in the one or more tumor cells and an amount of PTEN expression in the one or more normal cells;
(d) comparing the amount of PTEN expression in the tumor cells to the amount of PTEN expression in the normal cells; and
(e) determining that PTEN is elevated in the tumor cells where the amount of expression of PTEN is greater in the tumor cells as compared to the normal cells or determining that PTEN is reduced in the tumor cells where the amount of expression of PTEN is less in the tumor cells than in the normal cells.

2. The method of claim 1, wherein the normal cells are stromal cells.

3. The method of claim 1, wherein the PTEN is mutated.

4. The method of claim 1, wherein biological sample is a tumor biopsy.

5. The method of claim 1, wherein the biological sample is an aspirate.

6. The method of claim 1, wherein the normal and tumor cells are assayed using a detectably labeled antibody or binding fragment thereof specific for PTEN.

7. The method of claim 6, wherein the antibody is a monoclonal antibody.

8. The method of claim 6, wherein the label is a chromagen or fluorophore.

9. The method of claim 1, wherein the step of assaying for expression of PTEN is performed by immunohistochemistry (IHC) or western blot.

10. The method of claim 9, wherein the step of quantitating PTEN expression in the normal cells and the tumor cells is preformed by image analysis.

11. The method of claim 10, wherein the step of quantitating PTEN expression in the normal cells and the tumor cells is preformed in a defined cellular area.

12. The method of claim 11, wherein the defined cellular area is the nucleus.

13. The method of claim 11, wherein the defined cellular area is the cytoplasm.

14. The method of claim 11, wherein the defined cellular area is the membrane.

15. The method of claim 11, wherein the amount of PTEN expression for the normal cells and the amount of expression for the tumor cells is quantitated from an average optical density (OD) of PTEN per pixel of the defined cellular area.

16. The method of claim 11, wherein the amount of PTEN expression for the normal cells and the amount of expression for the tumor cells is quantitated from an average OD determined for the tumor cells and the normal cells on a per cell basis in the defined cellular area.

17. The method of claim 15, wherein the average OD on a per cell basis is obtained by dividing the average OD for the defined cellular area by a number of nuclei in the defined cellular area.

18. The method of claim 1, wherein PTEN comprises the amino acid sequence as set forth in SEQ ID NO: 1.

19. A method for determining if PTEN expression levels are elevated or reduced in one or more tumor cells in a biological sample, said method comprising:
a. obtaining a biological sample comprising one or more tumor cells and one or more stromal cells;
b. assaying the biological sample for expression of PTEN by using a detectably labeled binding molecule specific for PTEN;
c. quantitating an amount of PTEN expression in the one or more tumor cells and an amount of PTEN expression in the one or more stromal cells by determining an optical density (OD) reading for PTEN expression in the tumor cells and an OD reading for PTEN expression in the normal cells, wherein the OD reading for the tumor cells and stromal cells is quantitated from an average OD reading determined for the tumor cells and the normal cells on a per cell basis in a defined cellular area and wherein the average OD reading on a per cell basis is obtained by dividing the average OD reading for the defined cellular area by a number of nuclei in the defined cellular area;
d. comparing the PTEN OD reading in the tumor cells to the PTEN OD reading in the stromal cells; and
e. determining that PTEN is elevated in the tumor cells where the PTEN OD reading is greater in the tumor cells as compared to the stromal cells or determining that PTEN is reduced in the tumor cells where the PTEN OD reading is less in the tumor cells than in the stromal cells.

20. A method for predicting responsiveness of a subject with a disease or disorder to a receptor tyrosine kinase inhibitor, said method comprising:
a. obtaining the biological sample comprising one or more tumor cells and one or more normal cells from the subject;
b. assaying the biological sample for expression of PTEN;
c. quantitating an amount of PTEN expression in the one or more tumor cells and an amount of PTEN expression in the one or more normal cells;
d. comparing the amount of PTEN expression in the tumor cells to the amount of PTEN expression in the normal cells;
e. determining that PTEN is elevated in the tumor cells where the amount of expression of PTEN is greater in the tumor cells as compared to the normal cells or determining that PTEN is reduced in the tumor cells where the amount of expression of PTEN is less in the tumor cells than in the normal cells; and
f. predicting that the subject is responsive to a receptor tyrosine kinase inhibitor where expression of PTEN in the tumor cells is less than expression of PTEN in the normal cells or determining that the subject is not responsive to a receptor tyrosine kinase inhibitor where expression of PTEN in the tumor cells is the same or greater than expression of PTEN in the normal cells.

21. The method of claim 20, wherein the normal cells are stromal cells.

22. The method of claim 20, wherein the PTEN is mutated.

23. The method of claim 20, wherein biological sample is a tumor biopsy.

24. The method of claim 20, wherein the biological sample is an aspirate.

25. The method of claim 20, wherein the normal and tumor cells are assayed using a detectably labeled antibody or binding fragment thereof specific for PTEN.

26. The method of claim 25, wherein the antibody is a monoclonal antibody.

27. The method of claim 25, wherein the label is a chromagen or fluorophore.

28. The method of claim 20, wherein the step of assaying is performed by immunohistochemistry (IHC) or western blot.

29. The method of claim 28, wherein the step of quantitating PTEN expression in the normal cells and the tumor cells is preformed by image analysis.

30. The method of claim 29, wherein the step of quantitating PTEN expression in the normal cells and the tumor cells is preformed in a defined cellular area.

31. The method of claim 30, wherein the defined cellular area is the nucleus.

32. The method of claim 30, wherein the defined cellular area is the cytoplasm.

33. The method of claim 30, wherein the defined cellular area is the membrane.

34. The method of claim 30, wherein the amount of PTEN expression for the normal cells and the tumor cells is quantitated from an average optical density (OD) of PTEN per pixel of the defined cellular area.

35. The method of claim 30, wherein the amount of PTEN expression for the normal cells and the tumor cells is quantitated from an average OD determined for the tumor cells and the normal cells on a per cell basis in the defined cellular area.

36. The method of claim 35, wherein the average OD on a per cell basis is obtained by dividing the average OD for the defined cellular area by a number of nuclei in the defined cellular area.

37. The method of claim 20, wherein PTEN comprises the amino acid sequence as set forth in SEQ ID NO: 1.

38. The method of claim 20, wherein the receptor tyrosine kinase inhibitor is an antibody.

39. The method of claim 38, wherein the antibody is a monoclonal antibody.

40. The method of claim 39, wherein the monoclonal antibody is cetuximab (Erbitux), panitumumab, zalutumumab, nimotuzumab or matuzumab.

41. The method of claim 20, wherein the receptor tyrosine kinase inhibitor is a small molecule inhibitor.

42. The method of claim 41, wherein the small molecule inhibitor is gefitinib, erlotinib or lapatinib.

43. The method of claim 20, wherein the disease or disorder is cancer.

44. The method of claim 43, wherein the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, renal cancel, pancreatic cancer, genital-urinary cancer and bladder cancer.

45. The method of claim 20, wherein the subject is a cancer patient.

46. A method for treating a subject with a disease or disorder with a receptor tyrosine kinase inhibitor, the method comprising:
a. obtaining the biological sample comprising one or more tumor cells and one or more normal cells from the subject;
b. assaying the biological sample for expression of PTEN;
c. quantitating an amount of PTEN expression in the one or more tumor cells and an amount of PTEN expression in the one or more normal cells;
d. comparing the amount of PTEN expression in the tumor cells to the amount of PTEN expression in the normal cells;
e. determining that PTEN is elevated in the tumor cells where the amount of expression of PTEN is greater in the tumor cells as compared to the normal cells or determining that PTEN is reduced in the tumor cells where the amount of expression of PTEN is less in the tumor cells than in the normal cells; and
f. administering the receptor tyrosine kinase inhibitor to the subject.

47. The method of claim 46, wherein the normal cells are stromal cells.

48. The method of claim 46, wherein the PTEN is mutated.

49. The method of claim 46, wherein biological sample is a tumor biopsy.

50. The method of claim 46, wherein the biological sample is an aspirate.

51. The method of claim 46, wherein the normal and tumor cells are assayed using a detectably labeled antibody or binding fragment thereof specific for PTEN.

52. The method of claim 51, wherein the antibody is a monoclonal antibody.

53. The method of claim 51, wherein the label is a chromagen or fluorophore.

54. The method of claim 46, wherein the step of assaying is performed by immunohistochemistry (IHC) or western blot.

55. The method of claim 54, wherein the step of quantitating PTEN expression in the normal cells and the tumor cells is preformed by image analysis.

56. The method of claim 55, wherein the step of quantitating PTEN expression in the normal cells and the tumor cells is preformed in a defined cellular area.

57. The method of claim 56, wherein the defined cellular area is the nucleus.

58. The method of claim 56, wherein the defined cellular area is the cytoplasm.

59. The method of claim 56, wherein the defined cellular area is the membrane.

60. The method of claim 56, wherein the amount of PTEN expression for the normal cells and the tumor cells is quantitated from an average optical density (OD) of PTEN per pixel of the defined cellular area.

61. The method of claim 56, wherein the amount of PTEN expression for the normal cells and the tumor cells is quantitated from an average OD determined for the tumor cells and the normal cells on a per cell basis in the defined cellular area.

62. The method of claim 61, wherein the average OD on a per cell basis is obtained by dividing the average OD for the defined cellular area by a number of nuclei in the defined cellular area.

63. The method of claim 46, wherein PTEN comprises the amino acid sequence as set forth in SEQ ID NO: 1.

64. The method of claim 46, wherein the receptor tyrosine kinase inhibitor is an antibody.

65. The method of claim 64, wherein the antibody is a monoclonal antibody.

66. The method of claim 65, wherein the monoclonal antibody is cetuximab (Erbitux), panitumumab, zalutumumab, nimotuzumab or matuzumab.

67. The method of claim 46, wherein the receptor tyrosine kinase inhibitor is a small molecule inhibitor.

68. The method of claim 67, wherein the small molecule inhibitor is gefitinib, erlotinib or lapatinib.

69. The method of claim 46, wherein the disease or disorder is cancer.

70. The method of claim 69, wherein the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

71. The method of claim 46, wherein the subject is a cancer patient.

72. A method for selecting subjects for inclusion/exclusion in a clinical trial, said method comprising:
a. obtaining a biological sample from each subject comprising one or more tumor cells and one or more stromal cells;
b. assaying the biological sample obtained from each subject for expression of PTEN by using a detectably labeled binding molecule specific for PTEN;
c. quantitating an amount of PTEN expression in the one or more tumor cells and an amount of PTEN expression in the one or more stromal cells;
d. comparing the amount of PTEN expression in the tumor cells to the amount of PTEN expression in the stromal cells;
e. determining that PTEN expression levels are elevated in the tumor cells where the amount of PTEN expression is greater in the tumor cells as compared to the stromal cells or determining that PTEN expression is reduced in the tumor cells where the amount of PTEN expression is less in the tumor cells than in the stromal cells; and
f. selecting subjects for inclusion/exclusion in the clinical trial that have an amount of PTEN expression that is greater in the tumor cells as compared to the stromal cells or selecting subjects for inclusion/exclusion in the clinical trial that have an amount of PTEN expression that is less in the tumor cells as compared to the stromal cells.

73. The method of claim 72, wherein the clinical trial is a phase I, phase II, phase III or phase IV clinical trial.

74. The method of claim 72, wherein subjects are selected for inclusion in the clinical trial that have an amount of PTEN expression that is greater in the tumor cells as compared to the stromal cells.

75. The method of claim 72, wherein subjects are selected for exclusion from the clinical trial that have an amount of PTEN expression that is greater in the tumor cells as compared to the stromal cells.

76. The method of claim 72, wherein subjects are selected for inclusion in the clinical trial that have an amount of PTEN expression that is equal to or less than in the tumor cells as compared to the stromal cells.

77. The method of claim 72, wherein subjects are selected for exclusion from the clinical trial that have an amount of PTEN expression that is equal to or less than in the tumor cells as compared to the stromal cells.
